(19) United States
(12) Patent Application Publication
(10) Pub. No.: US 2008/0208299 A1
(43) Pub. Date: Aug. 28, 2008

(54) THERMO-THERAPEUTIC CUSHION

(76) Inventor: Sylvain Martineau, Drummondville (CA)

Correspondence Address: BERESKIN AND PARR, 40 KING STREET WEST, BOX 401, TORONTO, ON, M5H 3Y2, omitted

(21) Appl. No.: 11/680,137

(22) Filed: Feb. 28, 2007

(51) Int. Cl. A61F 7/08

(52) U.S. Cl. 607/112; 607/96

(57) ABSTRACT

A microwaveable thermo-therapeutic cushion for placing on a body is disclosed. The thermo-therapeutic cushion comprises a flexible closed shell partially filled with a plurality of legume seeds. The plurality of legume seeds are operative to store heat upon being subjected to microwaves from a microwave oven and slowly release it to the body through the shell. The legume seeds may be lentils.

### FIELD OF THE INVENTION

The present invention generally relates to the field of therapeutic equipment. More specifically, the invention relates to a thermo-therapeutic cushion for transferring heat to an external part of a body.

### BACKGROUND OF THE INVENTION

Providing heat to an aching human body part is a known method of pain relief. Long time ago, people would use a heated stone, wrap it in a piece of leather and place the leather against the aching body part. More recently, thermo-therapeutic cushions have been introduced to perform the same task. The principle, however, remains always the same: a material capable of storing heat is wrapped inside an envelope that somewhat isolates the heated material, thereby slowly conducting heat to the contacting body part. Because they are flexible and contain cereal grains, these newer thermo-therapeutic cushions are convenient as they conform well to different body parts. They are also easier to heat than the stones of the old ages.

Such thermo-therapeutic cushions have been in use for a while. For example, U.S. Pat. No. 5,300,104 to Gaudreault discloses a flexible thermo-therapeutic pad that uses oats with precise water content. The oats is contained in a fabric envelope and is heatable in a microwave oven. Using oats, however, has some drawbacks: many people complain of the smell of oats when heated. Furthermore, although oats performs reasonably well, it would always be beneficial to find a type of particle having a higher thermal inertia, thereby being capable of releasing its heat over a longer period of time.

There is therefore a need for a means to provide heat to a human body part that does not smell oats when heated and that transfers its heat over an extended period of time.

### SUMMARY OF THE INVENTION

It is an object of the present invention to provide a thermo-therapeutic cushion that overcomes or mitigates one or more disadvantages of known thermo-therapeutic cushions, or at least provides a useful alternative.

The present invention provides the advantages of being capable of providing heat to a human body part while not releasing a smell of heated oats. Furthermore, the present invention provides the advantage of transferring its heat over an extended period of time.

In accordance with an embodiment of the present invention, there is provided a microwaveable thermo-therapeutic cushion for placing on a body. The thermo-therapeutic cushion comprises a flexible closed shell and a plurality of legume seeds enclosed in the shell. The plurality of legume seeds are operative to store heat upon being subjected to microwaves from a microwave oven and slowly release it to the body through the shell. The shell is partially filled with the plurality of legume seeds so as to remain flexible.

In accordance with an aspect of the present invention, the legume seeds may be lentils. The shell may be made of a fabric such as cotton. The shell is preferably filled between 60% and 85% by volume with legume seeds. More preferably, the shell is filled at 75% by volume with legume seeds.

In accordance with another aspect of the invention, the cushion may be equipped with a strap attached to the shell for securing the cushion over the body. The strap may be stretchable. The strap may comprises attaching means at each end of the strap for conveniently attaching its ends together.

### BRIEF DESCRIPTION OF DRAWINGS

These and other features of the present invention will become more apparent from the following description in which reference is made to the appended drawings wherein:

FIG. 1 is a perspective view of a thermo-therapeutic cushion in accordance with an embodiment of the present invention.

FIG. 2 is a perspective view of the thermo-therapeutic cushion of FIG. 1 in use.

FIG. 3 is a perspective view of a thermo-therapeutic cushion in accordance with another embodiment of the present invention.

FIG. 4 is a perspective view of a thermo-therapeutic cushion in accordance with another embodiment of the present invention.

### DETAILED DESCRIPTION OF THE INVENTION

In the present description of the invention, the term legume seed is understood to mean a legume seed that is in naturally dried, mature state. This is by opposition to a legume seed that would be in its fresh, immature state.

The present invention relates to a microwaveable thermo-therapeutic cushion capable of transferring heat so as to soothe an aching body part of a person by placing the cushion against the body part. The cushion is made of a closed shell containing in its inside volume a plurality of legume seeds having a thermal inertia that allows them to store heat and slowly release it to the body part.

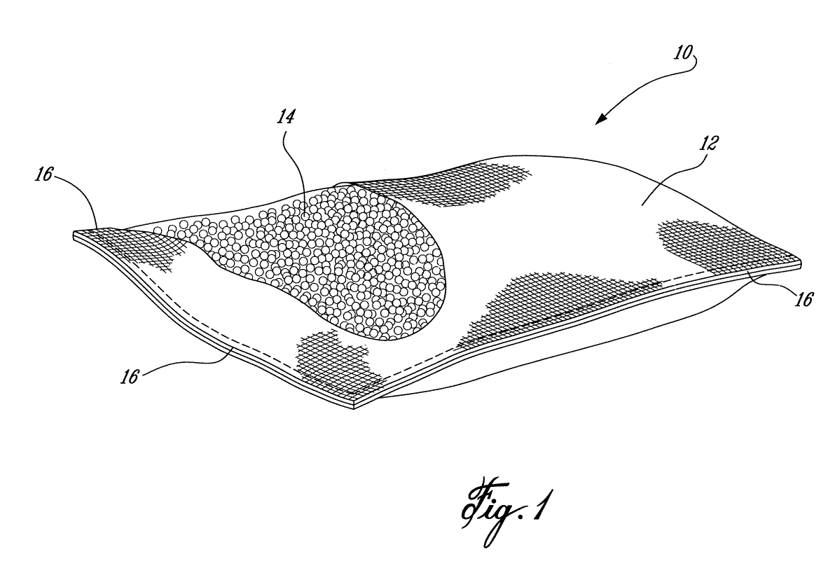

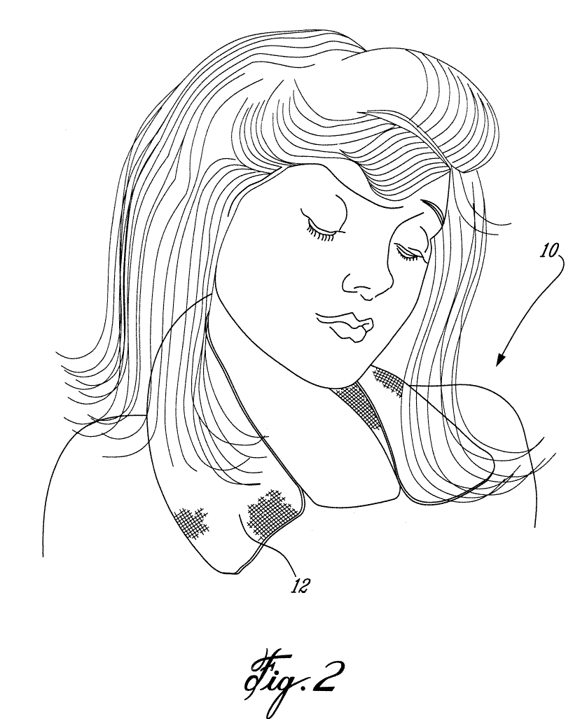

FIG. 1 depicts a first embodiment of the invention. A thermo-therapeutic cushion 10, looking much like a bag, is made of a closed shell 12 containing a plurality of naturally dried legume seeds 14. The closed shell 12 is heat-resistant and flexible. The closed shell 12 may be manufactured by cutting a rectangular piece of cloth, folding it in two, and sewing together both layers on its open edges 16. The closed shell 12 may be made in any other convenient way. The closed shell 12 may also take many shapes or forms to better suit certain parts of a human body. FIG. 2, which is concurrently referred to, shows an example of the thermo-therapeutic cushion 10, where the closed shell 12 may be narrow and elongated to better suit a neck and shoulders area. Similarly, the closed shell 12 could be shaped more like a square to better fit a lumbar area. It may also be smaller to fit smaller body parts, such as a hand, for example. The closed shell 12 must be made of a heat resistant material as it is in contact with the legume seeds 14, which release heat during use. If air is present inside the closed shell 12, the shell material may be porous so as not to explode from internal pressure created from the expansion of air when heated. The shell material must not have a high heat conductivity (such as aluminum, for example), which would risk burning the body parts. On the other hand, the shell material must not have a too low heat-conductivity either, which would impair the heat transfer from the legume seeds to the body part. The closed shell 12 must also be flexible so as to conform to the different body parts. Certain types of fabrics have been successfully used as a shell material. Preferably, 100% natural fibers materials, such as cotton or fleece are used.

The legume seeds 14 must be of a type having a relatively large thermal inertia in order to store heat and slowly release it to the body part. The legume seeds 14 must be dry so as not to stick together and not to rot. Therefore, legume seeds 14 must be used in their dry, mature state rather than in their fresh, immature state. The closed shell 12 must only be partially filled with legume seeds 14 so as to allow a free flow of the legume seeds 14 inside the closed shell 12. Typically, the closed shell 12 is filled by volume between 60% and 85% with the legume seeds 14. It has been found that filling the closed shell 14 to 75% of the volume provides good results. The free flow of legume seeds 14, combined with the fact that the shell 12 is flexible, allow the thermo-therapeutic cushion 10 to conform to the shape of the body part.

Different types of legumes seeds 14 may be used. For example, it has been found that using lentils as the legume seeds 14 provided good results.

In use, the thermo-therapeutic cushion 10 is placed 2 minutes at maximum power in a microwave oven. The thermo-therapeutic cushion 10 is then removed from the oven and placed over a body part. Alternatively, the thermo-therapeutic cushion 10 may also be placed in a cold place, such as a refrigerator or a freezer. Similarly, the thermo-therapeutic cushion 10 is removed from the cold place and placed on a body part for treatment with cold.

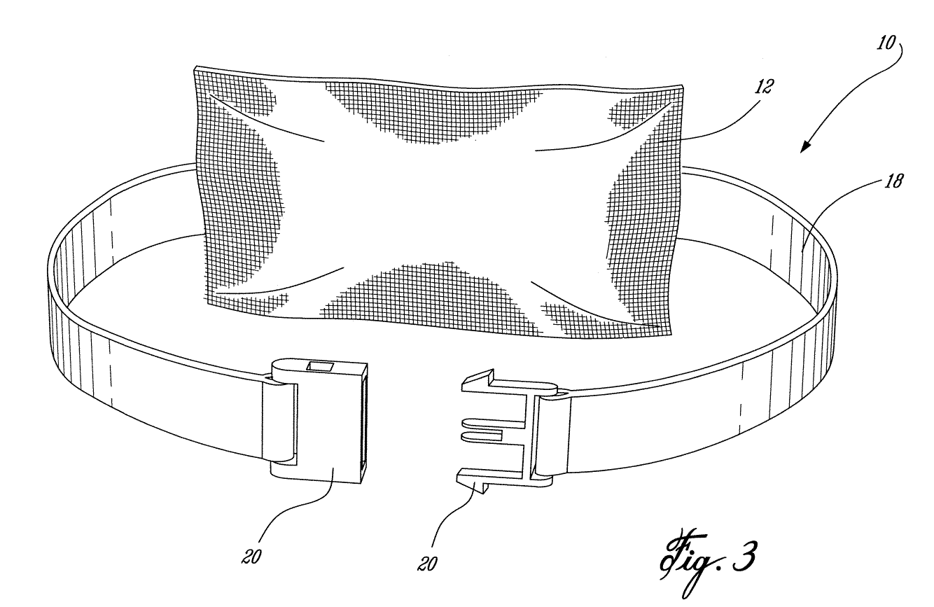

Turning now to FIG. 3, there is depicted another embodiment of the invention where the thermo-therapeutic cushion 10 is equipped with a strap 18 in order to secure the thermo-therapeutic cushion 10 over a body part. The strap 18 may be made of two parts, sewn to opposed ends of the thermo-therapeutic cushion 10, or be of a single part, sewn across the shell 12. The strap 18 may be stretchable so as to stretch over a body part, such as a head. Alternatively, the strap 18 may be divided in two half-straps 19 and equipped with attaching means 20 at each free end of the half-straps 18.

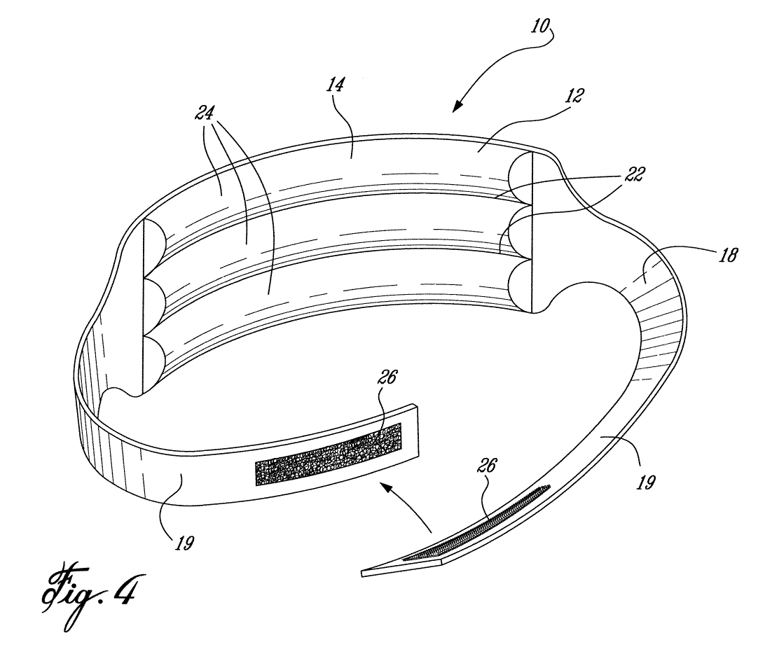

FIG. 4 depicts a variant of a strap-equipped thermo-therapeutic cushion. In this embodiment, the strap 18 is integrally made with the shell 12. The shell 12 is horizontally sown on itself at two locations 22 in order to create three thinner horizontal tubes 24, thereby preventing the legume seeds 14 inside the shell 12 to run all the way to the bottom of the shell 12 when the thermo-therapeutic cushion 10 is in working position such as across the back of the user, for example. Each half-strap 19 is equipped with matching bands 26 of Velcro®, which may be adjusted to fit snugly over a body part of the user.

The present invention has been described with regard to preferred embodiments. The description as much as the drawings were intended to help the understanding of the invention, rather than to limit its scope. It will be apparent to one skilled in the art that various modifications may be made to the invention without departing from the scope of the invention as described herein, and such modifications are intended to be covered by the present description.

I claim:

1. A microwaveable thermo-therapeutic cushion for placing on a body comprising:
a flexible closed shell; and
a plurality of legume seeds partially filling said shell,
wherein said plurality of legume seeds are operative to store heat upon being subjected to microwaves from a microwave oven and slowly release it to the body through said shell.

2. The cushion of claim 1 wherein said legume seeds are lentils.

3. The cushion of claim 2 wherein said shell is made of a fabric.

4. The cushion of claim 3 wherein said fabric is cotton.

5. The cushion of claim 4 wherein a volume inside said shell is filled between 60% and 85% by volume with said plurality of legume seeds.

6. The cushion of claim 5 wherein said volume inside said shell is filled at 75% by volume with said plurality of legume seeds.

7. The cushion of claim 1 wherein said shell is porous.

8. The cushion of claim 1 further comprising a strap attached to said shell for securing said cushion over the body.

9. The cushion of claim 8 further wherein said strap is stretchable.

10. The cushion of claim 8 wherein said strap further comprises attaching means at each end of said strap for attaching said ends together.

11. The use of legume seeds in the manufacture of a microwaveable, flexible thermo-therapeutic cushion.

12. The use of legume seeds as defined in claim 11 wherein said legume seeds are lentils.
